(19) United States
(12) Patent Application Publication
(10) Pub. No.: US 2024/0065561 A1
(43) Pub. Date: Feb. 29, 2024

(54) LIMITED USE BLOOD PRESSURE CUFF WITH SELF-DEGRADING LOOP AND HOUSING FOR DISPENSING SAME FROM WALL

(71) Applicant: Welch Allyn, Inc., Skaneateles Falls, NY (US)

(72) Inventors: Aaron R. Burnham, Auburn, NY (US); Megan Mary Burkarski, Syracuse, NY (US); Carlos Suarez, Syracuse, NY (US); Timothy Braun, Union Springs, NY (US); James Cappon, Fayetteville, NY (US)

(21) Appl. No.: 18/237,559

(22) Filed: Aug. 24, 2023

Related U.S. Application Data
(60) Provisional application No. 63/401,010, filed on Aug. 25, 2022.

(51) Int. Cl. A61B 5/022; A61B 5/021

(52) U.S. Cl. CPC A61B 5/02233; A61B 5/02141

(57) ABSTRACT

A housing assembly includes a housing body that defines an internal chamber. A connection bracket is located in the internal chamber and includes a first spindle bracket and a second spindle bracket. The first spindle bracket has a first recess and the second spindle bracket has a second recess. A cuff roll assembly includes a first spindle located in the first recess and a second spindle located in the second recess. The first spindle is spaced from the second spindle. A roll of pressure cuffs includes a plurality of pressure cuffs that are sequentially connected and wrapped around the first spindle and the second spindle. The plurality of pressure cuffs each include a first connection strip that is formed of hooks and a second connection strip that is formed of loops. The loops are formed of non-woven polyethylene terephthalate spunlace material.

### CROSS-REFERENCE TO RELATED APPLICATION

This application claims priority to and the benefit under 35 U.S.C. § 119(e) of U.S. Provisional Application No. 63/401,010, filed on Aug. 25, 2022, entitled “LIMITED USE BLOOD PRESSURE CUFF WITH SELF-DEGRADING LOOP AND HOUSING FOR DISPENSING SAME FROM WALL,” the disclosure of which is hereby incorporated herein by reference in its entirety.

### FIELD OF THE DISCLOSURE

The present disclosure generally relates to limited use pressure cuffs and a housing assembly that stores and dispenses pressure cuffs, for example, the limited use pressure cuffs.

### SUMMARY OF THE DISCLOSURE

According to one aspect of the present disclosure, a housing assembly includes a housing body that defines an internal chamber for which at least one pressure cuff is located. A connection bracket is located in the internal chamber and includes a first spindle bracket and a second spindle bracket. The first spindle bracket has a first recess and the second spindle bracket has a second recess. A pivot connection is connected to the second spindle bracket and biases the second spindle bracket away from the first spindle bracket.

According to another aspect of the present disclosure, a housing assembly includes a housing body that defines an internal chamber. A connection bracket is located in the internal chamber and includes a first spindle bracket and a second spindle bracket. The first spindle bracket has a first recess and the second spindle bracket has a second recess. A cuff roll assembly includes a first spindle that is located in the first recess and a second spindle that is located in the second recess. The first spindle is spaced from the second spindle. A roll of pressure cuffs includes a plurality of pressure cuffs that are sequentially connected and wrapped around the first spindle and the second spindle.

According to another aspect of the present disclosure, a cuff roll assembly includes a first spindle that extends along a first axis and a second spindle that extends along a second axis that is spaced from the first axis. A roll of pressure cuffs includes a plurality of pressure cuffs that are sequentially connected and wrapped around the first spindle and the second spindle.

According to another aspect of the present disclosure, a roll of pressure cuffs includes a plurality of pressure cuffs that are integrally formed and sequentially connected. A first connection strip is formed of hooks on each pressure cuff of the plurality of pressure cuffs. A second connection strip is formed of loops on each of the pressure cuffs of the plurality of pressure cuffs. The loops are formed of non-woven polyethylene terephthalate spunlace material.

According to another aspect of the present disclosure, a roll of pressure cuffs includes a plurality of pressure cuffs. A first connection strip is formed of hooks on each pressure cuff of the plurality of pressure cuffs. A second connection strip is formed of loops on each of the pressure cuffs of the plurality of pressure cuffs. The loops are formed of non-woven polyethylene terephthalate spunlace material. The plurality of pressure cuffs are non-integrally formed and are sequentially connected by the first connection strip and the second connection strip.

According to another aspect of the present disclosure, a pressure cuff includes a first connection strip that is formed of hooks and a second connection strip that is formed of loops. The loops are formed of non-woven polyethylene terephthalate spunlace material.

These and other features, advantages, and objects of the present disclosure will be further understood and appreciated by those skilled in the art by reference to the following specification, claims, and appended drawings.

### BRIEF DESCRIPTION OF THE DRAWINGS

In the drawings:

FIG. 1 is a front perspective view of a housing assembly for storing and dispensing a plurality of pressure cuffs, according to an aspect of the present disclosure;

FIG. 2 is a front perspective view of a cuff roll assembly that facilitates storing and dispensing a plurality of pressure cuffs from a housing assembly, according to an aspect of the present disclosure;

FIG. 3 is a front perspective view of a cuff roll assembly illustrating a breakaway support that spaces a first spindle and a second spindle, according to an aspect of the present disclosure;

FIG. 4 is a front perspective view of a housing assembly where a cover and a cuff roll assembly have been removed, according to an aspect of the present disclosure;

FIG. 5 is a front elevational view of a housing assembly with a cuff roll assembly installed therein, according to an aspect of the present disclosure;

FIG. 6 is a top plan view of a limited use pressure cuff that may be installed on a cuff roll assembly, according to an aspect of the present disclosure;

FIG. 7 is a bottom plan view of a limited use pressure cuff, according to an aspect of the present disclosure; and

FIG. 8 is a side elevational view of a pair of limited use pressure cuffs, according to an alternative construction of the present disclosure.

### DETAILED DESCRIPTION

The present illustrated embodiments reside primarily in combinations of method steps and apparatus components related to limited use pressure cuffs and a housing assembly that stores and dispenses pressure cuffs, for example, the limited use pressure cuffs. Accordingly, the apparatus components and method steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

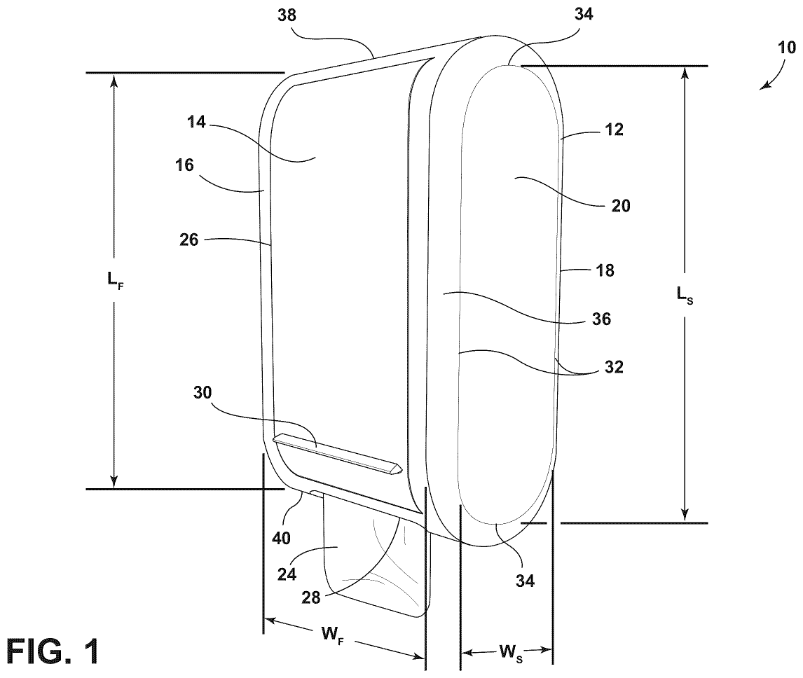

For purposes of description herein, the terms “upper,” “lower,” “right,” “left,” “rear,” “front,” “vertical,” “horizontal,” and derivatives thereof, shall relate to the disclosure as oriented in FIG. 1. Unless stated otherwise, the term “front” shall refer to a surface closest to an intended viewer, and the term “rear” shall refer to a surface furthest from the intended viewer. However, it is to be understood that the disclosure may assume various alternative orientations, except where expressly specified to the contrary. It is also to be understood that the specific structures and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The terms “including,” “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises a . . . ” does not, without more constraints, preclude the existence of additional identical elements in the process, method, article, or apparatus that comprises the element.

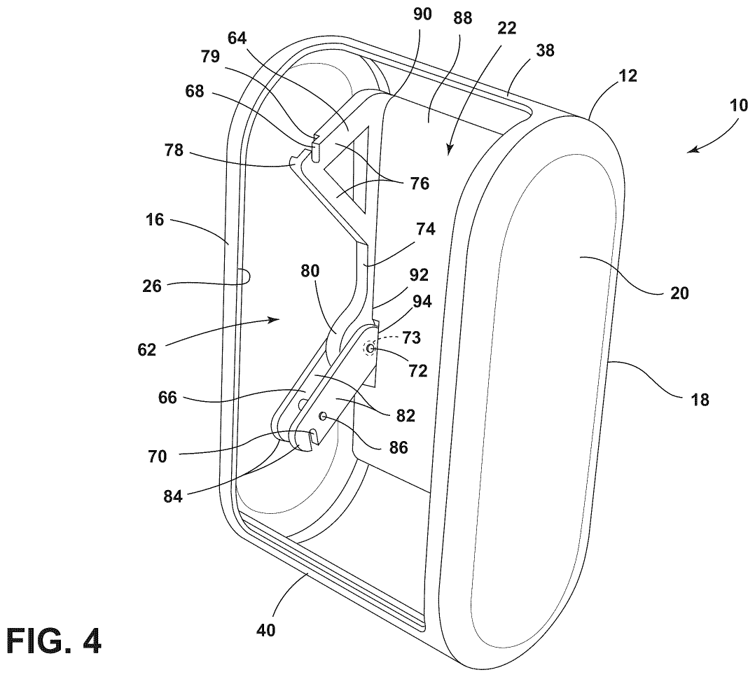

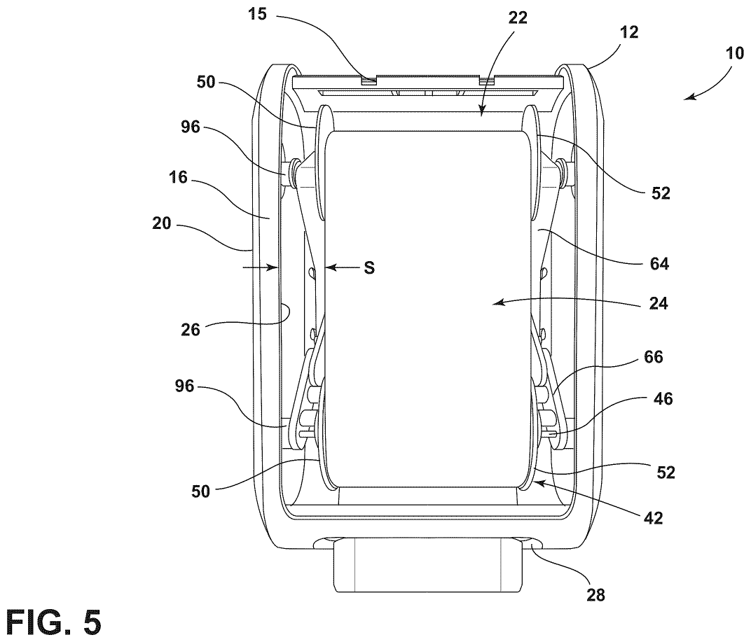

Referring initially to FIG. 1, reference numeral 10 generally designates a housing assembly. The housing assembly 10 includes a housing body 12 and a cover 14. The cover 14 is moveable relative to the housing body 12 between an open position and a closed position via at least one connector 15 (FIG. 5) located on the housing body 12 and/or the cover 14. For example, the connector 15 may include pins for pivotal connection, snap fit connections (e.g., to completely remove the cover 14), and/or a combination thereof. In some embodiments, the cover 14 is sectioned and can roll into the housing assembly 10 in a roller-type or sectional-type of configuration. The housing body 12 includes a front face 16 and a rear face 18 that are spaced by a pair of sidewalls 20. The front face 16, the rear face 18, and the sidewalls 20 together define an internal chamber 22 (FIGS. 4 and 5) for which at least one pressure cuff 24 (e.g., a plurality of pressure cuffs 24) is located. The front face 16 defines an opening 26 for which the cover 14 is located when in the closed position. In some embodiments, the cover 14 substantially obscures the opening 26. The front face 16 further defines a slot 28 from which the at least one pressure cuff 24 can be dispensed. In some embodiments, the slot 28 is located lower than (i.e., closer to a floor of an environment) that may be spaced from the opening 26. In other embodiments, the slot 28 may be coextensive with the opening 26 and at least partially defined by the cover 14. The cover 14 may include at least one grip 30, for example, an outwardly projecting lip that extends substantially across the cover 14 in a horizontal direction (i.e., substantially perpendicular to the sidewalls 20). In other embodiments, the grip 30 may be an inwardly extending recess or other configurations. The rear face 18 may connect to an installation surface of the environment wherein access to pressure cuffs is beneficial, for example, a wall in a medical facility. More particularly, the rear face 18 may define one or more apertures for receiving fasteners, adhesive pads, and/or the like.

With continued reference to FIG. 1, the sidewalls 20 may be oblong shaped. Each sidewall 20 includes an outer sidewall perimeter that includes two vertically extending linear sections 32 that are spaced by curved sections 34. In some embodiments, a chamfer 36 is located inwardly from the outer sidewall perimeter. The sidewalls 20 each include a sidewall width “Ws” and a sidewall length “Ls,” where the sidewall length Ls is greater than the sidewall width Ws. For example, the sidewall length Ls may be greater than the sidewall width Ws by a 2:1 ratio or more, a 3:1 ratio or more, or a 4:1 ratio or more. The front face 16 and rear face 18 may follow the outer sidewall perimeter, integrally, or otherwise connected from a top surface 38 to a bottom surface 40 that is configured to be lower than the top surface 38 when connected to the installation surface. The front face 16 and rear face 18 likewise define a face width “WF” and a face length “LF,” wherein the face length LF may be greater than or substantially equal to the sidewall length Ls. In some embodiments, the face width WF is greaterthan the than the sidewall width Ws. For example, the face width WF may be greater than the sidewall width Ws by a 2:1 ratio or more, a 3:1 ratio or more, or a 4:1 ratio or more. As such, when the housing assembly 10 is connected to the installation surface, the housing assembly 10 conforms closely with the installation surface for minimizing space usage requirements within the environment.

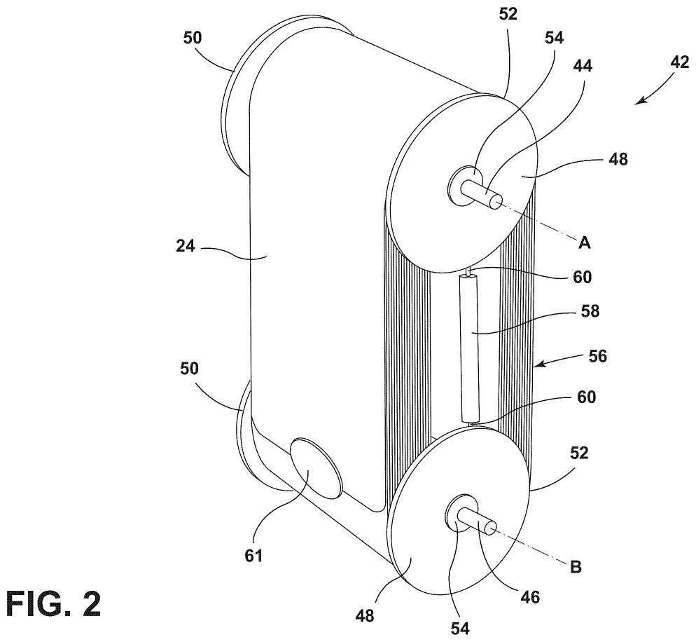

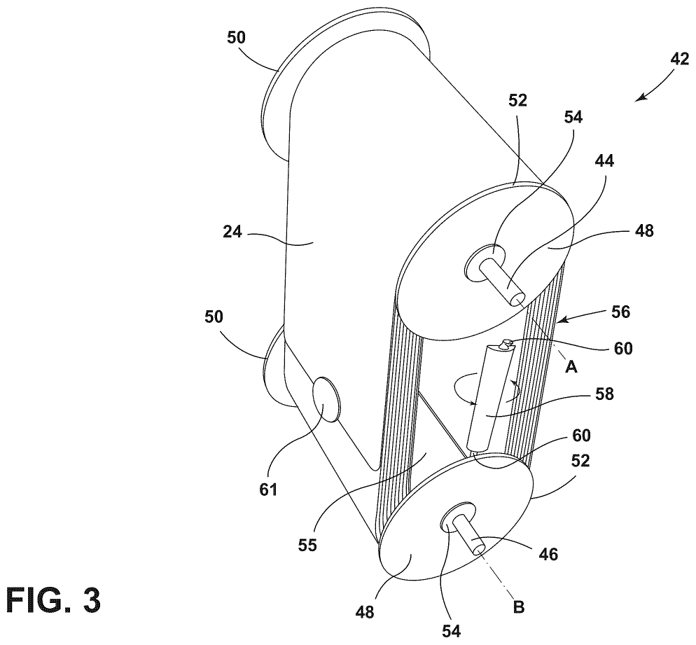

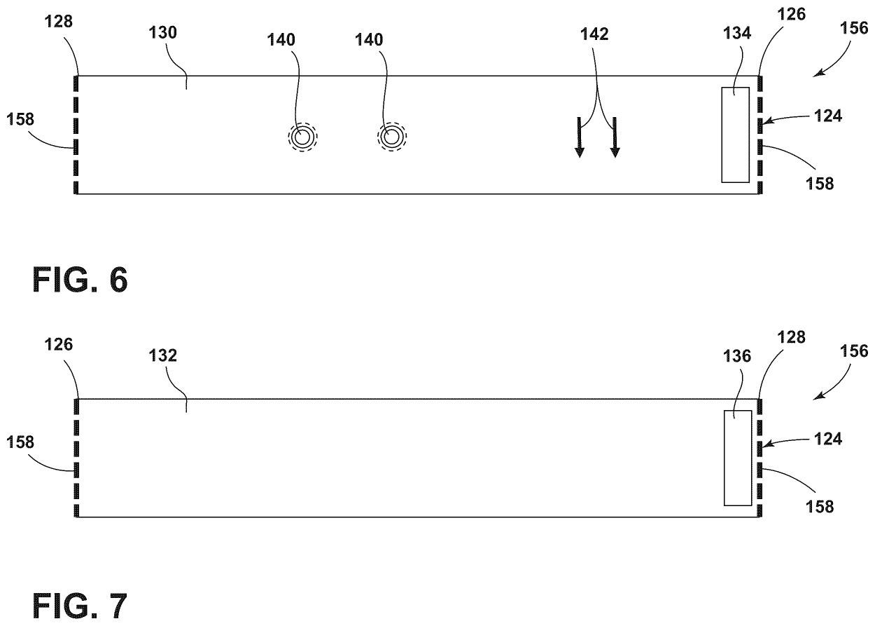

With reference now to FIGS. 2 and 3, the housing assembly 10 includes a cuff roll assembly 42 that facilitates storing and dispensing the plurality of pressure cuffs 24 from the housing assembly 10. The cuff roll assembly 42 may be configured to be replaceable, such that a plurality of cuff roll assemblies 42 can be installed and removed from the internal chamber 22. For example, when in use, the cuff roll assemblies 42 may be exchanged once the pressure cuffs 24 have been depleted and/or if different size or configurations of pressure cuffs 24 are needed. The cuff roll assembly 42 includes at least one spindle, for example, a first spindle 44 and a second spindle 46. The first spindle 44 may extend along a first axis A and the second spindle may extend along a second axis B that is spaced from the first axis A. For example, the first axis A and the second axis B may be configured to be spaced along the sidewall length Ls in a parallel relationship. Each of the first spindle 44 and the second spindle 46 may include a spool 48 that is integrally formed or connected for conjoint rotation. Each spool 48 may include a first flange 50 and a second flange 52 to axially retain the pressure cuffs 24 along the first axis A and the second axis B, respectively. A retaining ring 54 (e.g., a plurality of retaining rings 54) may connect the first and second spindles 44, 46 to the first and second flanges 50, 52, respectively. The retaining rings 54 may also function as a bearing surface during rotation of the spools 48. Each spool 48 may further include a body 55 (FIG. 3) that is sleeved around, otherwise connected, or formed integrally to the respective first and second spindles 44, 46. The body 55 may extend between the first flange 50 and the second flange 52, and interface with at least one pressure cuff 24. More particularly, the cuff roll assembly 42 may include a plurality of pressure cuffs 24 that define a roll of pressure cuffs 56 serially connected to one another. The connection of the pressure cuffs 24 in the roll of pressure cuffs 56 will be described in greater detail in reference to FIGS. 6-8. The roll of pressure cuffs 56 are wrapped around the first spindle 44 and the second spindle 46 and are retained with the spool 48. The cuff roll assembly 42 may further include at least one breakaway support 58 that operably connects the first spindle 44 to the second spindle 46 and is configured to be removed after installation of the cuff roll assembly 42 into the internal chamber 22. For example, the at least one breakaway support 58 (e.g., a pair of breakaway supports 58) may connect between the first flanges 50 and/or the second flanges 52 of the spools 48 on each of the first and second spindles 44, 46. The at least one breakaway support 58 spaces the first spindle 44 from the second spindle 46 and prevents relative rotation and movement therebetween until it is removed. The at least one breakaway support 58 may extend between opposite ends 60 of the breakaway support 58 that are configured to be detached from the first and second spindles 44, 46 and/or spools 48 via breaking or otherwise disconnecting. For example, the opposite ends 60 may break from the first and second spindles 44, 46 and/or spools 48 via rotation of the breakaway support 58. An end of a first pressure cuff 24 in the roll of pressure cuffs 56 may be connected to the roll of pressure cuffs 56 via an adhesive 61 (e.g., a tape, a glue, and/or the like).

With reference now to FIG. 4 the housing assembly 10 is illustrated with the cover 14 and the cuff roll assembly 42 removed. The housing assembly 10 may include a connection bracket 62 configured to connect and locate the cuff roll assembly 42 within the internal chamber 22. The connection bracket 62 may include a pair of connection brackets 62 adjacent to opposite sidewalls 20 (FIG. 5). For example, each connection bracket 62 may include a first spindle bracket 64 and a second spindle bracket 66. The first spindle bracket 64 may include a first recess 68 that cradles an end of the first spindle 44 and the second spindle bracket 66 may include a second recess 70 that cradles an end of the second spindle 46. In some embodiments, the first spindle recess 68 may open in a first direction and the second spindle recess 70 may open in a second direction that is substantially opposite to the first direction. Stated differently, the first spindle recess 68 may be oppositely oriented from the second spindle recess 70. For example, the first spindle recess 68 may open toward the top surface 38 and the second spindle recess 70 may open toward the bottom surface 40. At least one of the first spindle bracket 64 and the second spindle bracket 66 may be articulated with respect to the other of the first and second spindle brackets 64, 66. One of the first and second spindle brackets 64, 66 that can articulate may be biased away from the other of the first and second spindle brackets 64, 66. For example, the second spindle bracket 66 may be pivotally connected with respect to the housing body 12 via a pivot connection (e.g., a pivot pin 72). The second spindle bracket 66 is biased away from the first spindle bracket 64 with a biasing mechanism 73, such as a spring (e.g., a torsion spring that provides rotary force with respect to the pivot pin 72, a helical spring, other types of springs, or any of form of biasing mechanism). During installation, the first spindle 44 is located in the first recesses 68 of the opposing first spindle brackets 64 and the opposing second spindle brackets 66 are articulated against the bias toward the first spindle brackets 64 until the second spindle 46 can be located in the second recess 70. After the first spindle 44 and the second spindle 46 are located, the opposing second spindle brackets 66 are released and articulate away from the opposing first spindle brackets 64, and the roll of pressure cuffs 56 is tensioned. Accordingly, rolls of pressure cuffs 56 with various distances between the first and second spindles 44, 46 can be accommodated. In addition, as the number of pressure cuffs 24 are depleted in the roll of pressure cuffs 56, the biased articulation of the first spindle 44 from the second spindle 46 causes the roll of pressure cuffs 56 to remain tensioned via progressive articulation. It should be appreciated that the pivot connection could alternatively include various bearings, interlocked teeth, and/or the like, to effectuate pivotal movement. The breakaway support 58 may be detached prior to insertion of the cuff roll assembly 42. In some embodiments, the opening 26 is sized to permit the breakaway support 58 to be detached after to insertion of the cuff roll assembly 42 (FIG. 5).

With continued reference to FIG. 4, the first spindle bracket 64 may include a base member 74 that extends along the sidewall length Ls and a pair of first arms 76 that extend from the base member 74 merge at a tip 78. The first recess 68 may be adjacent to the tip 78 and may further include a depression section 79 to accommodate the retaining rings 54 as a bearing surface. The first arms 76 may extend transversely with respect to one another to form a triangular shape. The base member 74 extends past the first arms 76 to a connection end 80. The connection end 80 accommodates the pivot pin 72 for connection to the second spindle bracket 66. The base member 74 widens at the connection end 80, for example, the connection end 80 may be substantially circular in shape with the pivot pin 72 connected centrally thereto. The second spindle bracket 66 includes a pair of second arms 82 that extend substantially parallel to one another and that are located on opposite sides of the connection end 80 (e.g., opposite faces of the widened portions). The pair of second arms 82 are connected via the pivot pin 72 for co-rotation therewith or rotation relative thereto. The pair of second arms 82 each extend to ends 84, opposite the pivot pin 72, which define the second recess 70. The pair of second arms 82 may be further connected via one or more connection pins 86 located between the pivot pin 72 and the ends 84. The housing assembly 10 may further include a support plate 88 that extends substantially along the rear face 18 in the internal chamber 22. The support plate 88 provides additional structural integrity to the housing assembly 10 and may be integral with one or more portions of the base member 74. Alternatively, the support plate 88 may include opposite side edges 90 that conform to the connection bracket 62, for example, the opposite side edges 90 may include a first pocket 92 that accommodates the base member 74 and a second pocket 94 that accommodates the connection end 80 and the pivotal movement of the pair of second arms 82. In some embodiments, the first pocket 92 is coextensive with the second pocket 94.

With reference now to FIG. 5, the housing assembly 10 is illustrated with the cuff roll assembly 42 installed therein. When installed, the first flange 50 and the second flange 52 define a space “S” from the sidewalls 20, respectively, such that a user can manually disconnect the at least one breakaway support 58. The space S may be 1 cm or more, 2 cm or more, 3 cm or more, or 5 cm or less. The ends of the first spindle 44 and the second spindle 46 may be provided with caps 96 that limit movement of the first spindle 44 along the first axis A and the second spindle 46 along the second axis B. The caps 96 may further be shaped to at least partially extend over the first recess 68 and the second recess 70 to retain the first and second spindles 44, 46 therein. In this manner, pressure cuffs 24 of various sizes (e.g., widths) can be accommodated into the housing assembly 10. In some embodiments, the roll of pressure cuffs 56 are configured to be released in a clockwise direction with respect to the first axis A and the second axis B.

With reference now to FIGS. 6 and 7, a second embodiment of a pressure cuff 124 that may be installed on the cuff roll assembly 42, described above, is illustrated. Unless otherwise explicitly stated, the pressure cuff 124 according to the second embodiment may share all of the same features, functions, elements, and constructions of other embodiments described herein. Likewise, the pressure cuff 24 previously described, unless otherwise explicitly stated, may share all the same features, functions, elements, and constructions that will be described with the pressure cuff 124. The pressure cuff 124 may be of limited use such that it can only be used one or more times, two or more times, three or more times, five or less times, four or less times, three or less times, two or less times, or limited to a single use before it is discarded. The limited use configuration of the pressure cuff 124 may be beneficial in medical environments where limiting vectors of hospital acquired infection is beneficial as the pressure cuff 124 is discarded before it can be used between different (e.g., multiple) patients or the same patient over a period of time. More particularly, each of the pressure cuffs 124 includes an outer perimeter that extends between a first end 126 and a second end 128. Each of the pressure cuffs 124 further include a first side 130 and a second side 132. A first connection strip 134 extends adjacent to the first end 126 on the first side 130 and a second connection strip 136 extends adjacent to the second end 128 on the second side 132. The first connection strip 134 is configured to be releasably connected to the second connection strip 136. For example, one of the connection strips 134, 136 may include a loop structure (e.g., non-woven polyethylene terephthalate spunlace loops, non-woven material, spunlace loops, polyethylene terephthalate material, combinations thereof, and/or the like) and the other of the connection strips 134, 136 may include a hook structure (e.g., nylon hooks). The connection strips 134, 136 are configured to quickly degrade after usage, for example, by limited elastic memory of the hooks or other connection structures. For example, when the loop structures are formed of non-woven polyethylene terephthalate spunlace constructions rather than traditional nylon, it will quickly degrade after the limited number of uses described above. Each of the pressure cuffs 124 further include at least one port 140 for connecting at least one of an inflation member (e.g., an inflation bulb) and a pressure gauge. Each of the pressure cuffs 124 may further include an indicia 142 for aligning with a patient artery. When the pressure cuffs 124 are in a roll of pressure cuffs 156, ends 126, 128 of adjacent pressure cuffs 124 are releasably connected to one another. For example, the ends 126, 128 may be connected via perforation lines 158 that can be torn. As such, each pressure cuff 124 in the roll of pressure cuffs 156 may be integrally constructed.

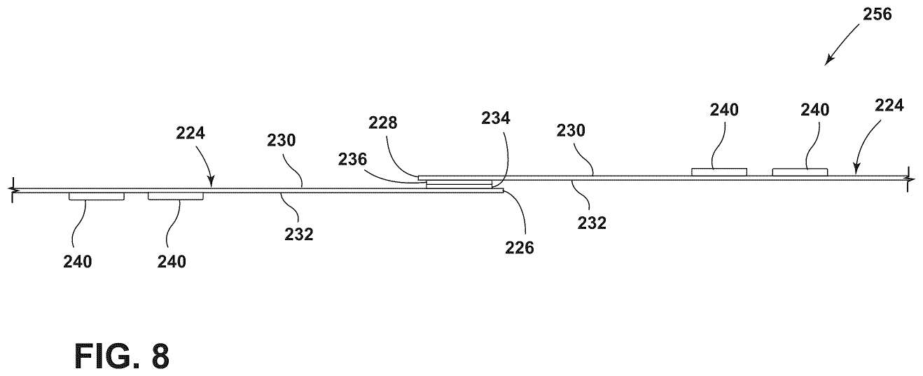

Referring now to FIG. 8, a third embodiment of a pair of pressure cuffs 224 that may be installed on the cuff roll assembly 42, described above, is illustrated. Unless otherwise explicitly stated, the pressure cuff 224 according to the third embodiment may share all of the same features, functions, elements, and constructions of other embodiments described herein. Likewise, the pressure cuffs 24, 124 previously described, unless otherwise explicitly stated, may share all the same features, functions, elements, and constructions that will be described with the pressure cuff 224. The pressure cuff 224 may be of limited use such it can only be used one or more times, two or more times, three or more times, five or less times, or limited to a single use before it is discarded. The limited use configuration of the pressure cuff 224 may be beneficial in medical environments wherein limiting vectors of hospital acquired infection is beneficial as the pressure cuff 224 is discarded before it can be used between multiple patients or a single patient over a period of time. More particularly, each of the pressure cuffs 224 includes an outer perimeter extending between a first end 226 and a second end 228. Each of the pressure cuffs 224 further include a first side 230 and a second side 232. A first connection strip 234 extends adjacent to the first end 226 on the first side 230 and a second connection strip 236 extends adjacent to the second end 228 on the second side 232. The first connection strip 234 is configured to be releasably connected to the second connection strip 236. For example, one of the connection strips 234, 236 may include a loop structure (e.g., non-woven polyethylene terephthalate spunlace loops, non-woven material, spunlace loops, polyethylene terephthalate material, combinations thereof, and/or the like) and the other of the connection strips 234, 236 may include a hook structure (e.g., nylon hooks). The connection strips 234, 236 are configured to quickly degrade after usage, for example, by limited elastic memory of the hook or other connecting structures. For example, when the loop structures are formed of non-woven polyethylene terephthalate spunlace constructions rather than traditional nylon, it will quickly degrade after a limited number of uses, as described above. Each of the pressure cuffs 224 further include at least one port 240 to connect at least one of an inflation member (e.g., an inflation bulb) and a pressure gauge. Each of the pressure cuffs 224 may further include an indicia to align with a patient's artery. When the pressure cuffs 224 are in a roll of pressure cuffs 256, ends 226, 228 of adjacent pressure cuffs 224 are releasably connected to one another. For example, the ends 226, 228 may be connected via the respective connection strips 234, 236 of adjacent pressure cuffs 224. As such, each pressure cuff 224 in the roll of pressure cuffs 256 may be non-integrally, or separately, constructed.

With reference to FIGS. 2, 3, and 6-8, the connection strips 134, 136, 234, 236 of the pressure cuffs 24, 124, and 224 may be located for additional functionalities. For example, the spacing may permit the connection strips 134, 234 of a single pressure cuff 24, 124, and 224 to connect to the other connection strips 136, 236 of the single pressure cuff 24, 124, and 224. In this manner, the connection strips 134, 136, 234, 236 may also be configured to connect during attachment and pressure readings on a patient in addition to facilitating the connection between adjacent pressure cuffs 24, 124, and 224 when in the housing assembly 10 and rolled into the cuff roll assembly 42.

According to one aspect of the present disclosure, a housing assembly includes a housing body that defines an internal chamber for which at least one pressure cuff is located. A connection bracket is located in the internal chamber and includes a first spindle bracket and a second spindle bracket. The first spindle bracket has a first recess and the second spindle bracket has a second recess. A pivot connection is connected to the second spindle bracket and biases the second spindle bracket away from the first spindle bracket.

According to another aspect of the present disclosure, a first recess opens in a first direction and a second recess opens in a second direction that is substantially opposite the first direction.

According to still another aspect of the present disclosure, a housing body defines a slot from which at least one pressure cuff can be dispensed.

According to another aspect of the present disclosure, a pivot connection includes a pivot pin.

According to yet another aspect of the present disclosure, a housing body includes a front face, a rear face, and a pair of sidewalls that space the front face from the rear face. Each sidewall of the pair of sidewalls defines a sidewall width. The front face defines a face width that is more than twice as large as the sidewall width.

According to still another aspect of the present disclosure, a connection bracket includes a pair of connection brackets that are located on opposite sides of an internal chamber.

According to another aspect of the present disclosure, at least one pressure cuff includes a first connection strip that is formed of hooks and a second connection strip that is formed of loops. The loops are formed of non-woven polyethylene terephthalate spunlace material.

According to yet another aspect of the present disclosure, a housing assembly includes a cover that has an open position where the internal chamber can be accessed and a closed position where access to the internal chamber is obstructed.

According to another aspect of the present disclosure, a housing assembly includes a housing body that defines an internal chamber. A connection bracket is located in the internal chamber and includes a first spindle bracket and a second spindle bracket. The first spindle bracket has a first recess and the second spindle bracket has a second recess. A cuff roll assembly includes a first spindle that is located in the first recess and a second spindle that is located in the second recess. The first spindle is spaced from the second spindle. A roll of pressure cuffs includes a plurality of pressure cuffs that are sequentially connected and wrapped around the first spindle and the second spindle.

According to still another aspect of the present disclosure, a pivot connection connects to a second spindle bracket and biases the second spindle bracket away from a first spindle bracket.

According to another aspect of the present disclosure, a first spindle bracket and a second spindle bracket both include a spool for conjoint rotation therewith.

According to still another aspect of the present disclosure, each spool includes a first flange and a second flange to retain a roll of pressure cuffs.

According to another aspect of the present disclosure, a retaining ring is located on each of a first flange and a second flange on an opposite side from a roll of pressure cuffs.

According to still another aspect of the present disclosure, a first recess defines a depression section to accommodate a retaining ring of a first spindle.

According to another aspect of the present disclosure, a housing assembly includes a breakaway support that is connected between a first spindle and a second spindle. The breakaway support is configured to be detached from each of the first spindle and the second spindle.

According to yet another aspect of the present disclosure, a plurality of pressure cuffs each include a first connection strip that is formed of hooks and a second connection strip that is formed of loops. The loops are formed of non-woven polyethylene terephthalate spunlace material.

According to another aspect of the present disclosure, a cuff roll assembly includes a first spindle that extends along a first axis and a second spindle that extends along a second axis that is spaced from the first axis. A roll of pressure cuffs includes a plurality of pressure cuffs that are sequentially connected and wrapped around the first spindle and the second spindle.

According to another aspect of the present disclosure, a cuff roll assembly includes a breakaway support that is connected between a first spindle and a second spindle. The breakaway support is configured to be detached from each of the first spindle and the second spindle.

According to still another aspect of the present disclosure, a first spindle and a second spindle both include a spool for conjoint rotation therewith.

According to another aspect of the present disclosure, a roll of pressure cuffs includes a plurality of pressure cuffs that are integrally formed and sequentially connected. A first connection strip is formed of hooks on each pressure cuff of the plurality of pressure cuffs. A second connection strip is formed of loops on each of the pressure cuffs of the plurality of pressure cuffs. The loops are formed of non-woven polyethylene terephthalate spunlace material.

According to still another aspect of the present disclosure, a plurality of pressure cuffs are sequentially connected with perforation lines.

According to another aspect of the present disclosure, a roll of pressure includes a plurality of pressure cuffs. A first connection strip is formed of hooks on each pressure cuff of the plurality of pressure cuffs. A second connection strip is formed of loops on each of the pressure cuffs of the plurality of pressure cuffs. The loops are formed of non-woven polyethylene terephthalate spunlace material. The plurality of pressure cuffs are non-integrally formed and are sequentially connected by the first connection strip and the second connection strip.

According to another aspect of the present disclosure, a pressure cuff includes a first connection strip formed of hooks and a second connection strip formed of loops. The loops are formed of non-woven polyethylene terephthalate spunlace material.

According to still yet another aspect of the present disclosure, a pressure cuff extends between opposite ends of the pressure cuff and each end includes a perforation line.

It will be understood by one having ordinary skill in the art that construction of the described disclosure and other components is not limited to any specific material. Other exemplary embodiments of the disclosure disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the disclosure, as shown in the exemplary embodiments, is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts, or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present disclosure. The exemplary structures and processes disclosed herein are for illustrative purposes and are not to be construed as limiting.

What is claimed is:

1. A housing assembly comprising:
a housing body defining an internal chamber for locating at least one pressure cuff;
at least one connection bracket located in the internal chamber and including a first spindle bracket and a second spindle bracket, wherein the first spindle bracket has a first recess and the second spindle bracket has a second recess; and
a pivot connection connected to the second spindle bracket and biasing the second spindle bracket away from the first spindle bracket.

2. The housing assembly of claim 1, wherein the first recess opens in a first direction and the second recess opens in a second direction that is substantially opposite to the first direction.

3. The housing assembly of claim 1, wherein the housing body defines a slot from which the at least one pressure cuff can be dispensed.

4. The housing assembly of claim 1, wherein the pivot connection includes a pivot pin.

5. The housing assembly of claim 1, wherein the housing body includes a front face, a rear face, and a pair of sidewalls spacing the front face from the rear face, wherein each of the sidewalls of the pair of sidewalls defines a sidewall width, and wherein the front face defines a face width that is more than twice as large as the sidewall width.

6. The housing assembly of claim 1, wherein the at least one connection bracket includes a pair of connection brackets located on opposite sides of the internal chamber.

7. The housing assembly of claim 1, wherein the at least one pressure cuff comprises:
a first connection strip formed of hooks; and
a second connection strip formed of loops, wherein the loops are formed of non-woven polyethylene terephthalate spunlace material.

8. The housing assembly of claim 1, further comprising:
a cover having an open position for accessing the internal chamber and a closed position for obstructing access to the internal chamber.

9. A housing assembly comprising:
a housing body defining an internal chamber;
a connection bracket located in the internal chamber and including a first spindle bracket and a second spindle bracket, wherein the first spindle bracket has a first recess and the second spindle bracket has a second recess;
a cuff roll assembly including a first spindle located in the first recess and a second spindle located in the second recess, wherein the first spindle is spaced from the second spindle; and
a roll of pressure cuffs including a plurality of pressure cuffs sequentially connected and wrapped around the first spindle and the second spindle.

10. The housing assembly of claim 9, further comprising:
a pivot connection connected to the second spindle bracket and biasing the second spindle bracket away from the first spindle bracket.

11. The housing assembly of claim 9, wherein the first spindle and the second spindle both include a spool for conjoint rotation with the first spindle and the second spindle.

12. The housing assembly of claim 11, wherein each spool includes a first flange and a second flange for retaining the roll of pressure cuffs.

13. The housing assembly of claim 12, wherein a retaining ring is located on each of the first flange and the second flange on an opposite side from the roll of pressure cuffs.

14. The housing assembly of claim 13, wherein the first recess defines a depression section for accommodating the retaining ring of the first spindle.

15. The housing assembly of claim 9, further comprising:
a breakaway support connected between the first spindle and the second spindle, wherein the breakaway support is configured to be detached from each of the first spindle and the second spindle.

16. The housing assembly of claim 9, wherein the plurality of pressure cuffs each comprise:
a first connection strip formed of hooks; and
a second connection strip formed of loops, wherein the loops are formed of non-woven polyethylene terephthalate spunlace material.

17. A cuff roll assembly comprising:
a first spindle extending along a first axis;
a second spindle extending along a second axis that is spaced from the first axis; and
a roll of pressure cuffs including a plurality of pressure cuffs sequentially connected and wrapped around the first spindle and the second spindle.

18. The cuff roll assembly of claim 17, wherein the plurality of pressure cuffs each comprise:
a first connection strip formed of hooks;
a second connection strip formed of loops; and
the hooks of one pressure cuff being connected to the loops of an adjacent pressure cuff in the roll of pressure cuffs forming the sequential connection.

19. The cuff roll assembly of claim 18, wherein the loops are formed of non-woven polyethylene terephthalate spunlace material.

20. The cuff roll assembly of claim 17, wherein each of the pressure cuffs is connected to an adjacent pressure cuff by a perforation line.
